(19) United States
(12) Patent Application Publication
(10) Pub. No.: US 2026/0096901 A1
(43) Pub. Date: Apr. 9, 2026

(54) HIGH-PROFILE, ANATOMY-SPECIFIC IMPLANTS FOR COMBINED HARD AND SOFT TISSUE RECONSTRUCTION WITH EMBEDDED TECHNOLOGY FOR MEDICINE DELIVERY

(71) Applicant: CraniUS LLC, Baltimore, MD (US)

(72) Inventors: Owen FRIESEN, Baltimore, MD (US); Ryan M. ZIMMERMAN, Lutherville, MD (US); Bejan DARBANDI, Baltimore, MD (US)

(73) Assignee: CraniUS LLC, Baltimore, MD (US)

(21) Appl. No.: 19/343,130

(22) Filed: Sep. 29, 2025

Related U.S. Application Data
(63) Continuation of application No. 18/908,988, filed on Oct. 8, 2024, now abandoned.

(51) Int. Cl. A61F 2/44; A61F 2/30; A61F 2/32; A61F 2/38; A61F 2/40

(52) U.S. Cl. CPC A61F 2/441; A61F 2/32; A61F 2/38; A61F 2/40; A61F 2002/30548; A61F 2002/30581; A61F 2002/30668; A61F 2002/3068

(57) ABSTRACT

According to an embodiment a combined hard and/or soft tissue medical implant may be provided for orthopedic and neurosurgical management of disease. The medical implant may further include an integrated reservoir, embedded pump-assisted smart technology, a strategic high-profile port for easy and safe access just underneath the neighboring skin, and one or more embedded catheters to deliver medication or cellular therapies.

### CROSS-REFERENCE TO RELATED APPLICATIONS

This application is a continuation of U.S. patent application Ser. No. 18/908,988, filed on Oct. 8, 2024, the contents of which is hereby incorporated in its entirety by reference.

### BACKGROUND

Degenerative osteoarthritis, septic arthritis, spine/joint tumors, and inflammatory arthritis of the spine and joints are major burdens of musculoskeletal disease and currently managed with a variety of treatments, including oral medications/antibiotics, intra-articular injections with pain medicines, radiation therapy, and surgical interventions including articular denervation, open or arthroscopic debridement, partial or total joint arthroplasty, spinal column resection with fusion or even limb amputation. Infection of a spinal implant or partial or complete joint replacement (periprosthetic joint infection) remains a common, challenging and costly clinical challenge, both in terms of patient morbidity and mortality, along with healthcare resources. Such infections often require implant removal, which is a devastating complication to the patient; especially when joint/spine symptoms persist and the infection and its symptoms prove unresponsive to systemic therapies like oral and intravenous antibiotics.

Diagnosis and management of periprosthetic joint infections is a vibrant area of research and innovation due to the extreme time, monetary expense and healthcare resources utilized for its management around the country. Current management includes diagnosis through a variety of imaging modalities and examination of blood, joint fluid and/or local tissue for the presence of or threshold values of certain cell types, bacterial strains, fungal species, and/or molecules. Management includes oral (enteral) and/or intravenous (parenteral) antibiotics or surgical intervention, including irrigation and debridement of the effected joint and either removal or retention of spine or arthroplasty components, based upon a variety of factors. Treatment often involves many weeks of costly, antimicrobial therapy and, in cases where arthroplasty components were removed, additional surgery to reconstruct the joint or spinal column, which is typically a more challenging undertaking due to bone loss or compromised soft tissues due to the infection and numerous surgical procedures. Thus, the orthopedic and neurosurgical markets are in dire need of a device for chronic medicine delivery to the local areas specific to the underlying problem, such as described above—to provide better options to spine and joint patients worldwide.

Native spine and joints can also be infected by a variety of microbial organisms (septic arthritis), often in immunocompromised individuals, and/or following trauma or medical interventions like surgery or biopsy. Though often less complicated to treat than a periprosthetic joint infection, one or more surgical interventions and prolonged oral or parenteral antibiotics are typically required, which again adds significant morbidity and expense to the healthcare system and patient.

### SUMMARY

According to an embodiment, a combined hard and/or soft tissue medical implant may be provided. The medical implant may further include an integrated reservoir, embedded pump-assisted technology, a high-profile injection port that may extend to a level just underneath the skin for easy and quick needle access, and one or more catheters to deliver medication into the spine and/or native joints, partial or complete joint replacement components, spinal implants, and/or a joint from which partial or total joint arthroplasty components have been removed due to infection or persistent pain.

In some embodiments the medical implant may be secured to neighboring soft tissue, bone, arthroplasty components, and/or may rest within the joint or prior arthroplasty space with either plate/screw fixation and/or suture fixation. In some embodiments the plate/screw may be, for example, titanium or another metal. In some embodiments the catheters may be directed into bone, soft tissue, native joint spaces, native spinal segments, and/or any soft or bony tissue containing a partial or total joint arthroplasty, or from which arthroplasty components or spinal implants have been removed, whereby the catheter tips may be strategically placed in areas where direct medicine delivery is critical and most productive to reverse underlying pathology in a way that mitigates the inefficiencies of oral and intravenous medicine delivery.

In other embodiments the catheters could also be directed into the components of any partial or total joint replacement surgical sites. Medications and cell therapies to be delivered may include, for example, tumoricidal medications, antibiotics, antifungals, anti-inflammatories, local anesthetics, performance enhancing medicines, regenerative cell therapy (i.e. stem cells) and/or other analgesics for chronic pain. The suggested device may be refillable by means of a transcutaneous injection port with a simple, non-boring needle. The device may also be either powered by passive conduction of an external wand, be rechargeable using direct application of a wireless recharging device, and/or be powered by using a long-distance, charging technology based on RF signals. The device may be both MRI safe and MRI compatible to allow uninterrupted imaging throughout the care cycle. Joints for treatment by the device may include but are not limited to the spine, shoulder, elbow, wrist, hip, knee and ankle. Spine areas for treatment by the device may include all levels of the spine including cervical, thoracic, lumbar, sacral and coccyx.

### BRIEF DESCRIPTION OF THE FIGURES

Advantages of embodiments of the present invention will be apparent from the following detailed description of the exemplary embodiments. The following detailed description should be considered in conjunction with the accompanying figures in which:

FIG. 1 shows an exemplary medical implant.

FIG. 2A shows an exemplary anatomical knee.

FIG. 2B shows an exemplary knee replacement implant.

FIG. 2C shows an exemplary lateral view of an anatomical knee.

FIG. 2D shows an exemplary lateral view of the knee replacement implant.

FIG. 3A shows an exemplary anatomical hip.

FIG. 3B shows an exemplary hip replacement implant.

FIG. 4A shows an exemplary anatomical shoulder.

FIG. 4B shows an exemplary shoulder replacement implant.

FIG. 5A shows an exemplary anatomical spine.

FIG. 5B shows an exemplary spine replacement implant.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

In one or more exemplary embodiment a medical implant device may be provided.

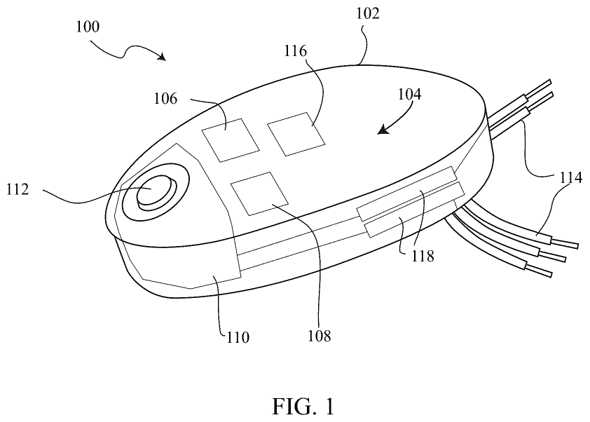

FIG. 1 shows an exemplary medical implant 100. The medical implant may be sized and shaped to fit within the spine or joint space, and further within embodiments of the extended soft tissue implant described herein. The medical implant 100 may include a housing 102, one or more electronic components 104, which may include a central processing unit 106 and a rechargeable battery 108. The medical implant 100 may further include a high-profile, refillable reservoir 110 having a cover or diaphragm 112 that may be penetrable by a percutaneous or similar needle and may strategically extend up to a level just beneath the skin. The medical implant 100 may further include a plurality of conduits or catheters 114, for example two catheters that may have length such that they can penetrate subcutaneously approximately 2-5 centimeters deep into surrounding spine or joint space. Additional electronic components disposed within functional component 100 may include, but are not limited to, a short range wireless module 116 such as a Bluetooth module, at least one propellant-driven or electro-osmotic pump 118, Furthermore, rechargeable battery 108 may utilize wireless charging so as to be able to charge from a distance, for example up to 18 inches away from medical implant 100 (i.e., the charging portion may be placed on the nightstand if and when the patient needs device-charging overnight, or within a garment or joint-specific covering (i.e. brace, shirt, pants) having internal components to allow charging during the daytime).

Furthermore, the cover or diaphragm 112 may protrude above the surrounding surface of housing 102, such that the diaphragm and “high-profile” design may be easily palpable under the skin to improve safety and efficacy of needle filling. It should be appreciated that this is in contrast to a “low-profile” interskeletal design, wherein the functional component would have a smooth contour with normal bone all around, and therefore not be palpable by one's fingers rubbing along the skin's surface, and presenting an impediment to percutaneous refilling of a reservoir. The medical implant 100, however, may extend in a high-profile manner so that the soft tissue implant is just beneath the skin, and therefore allows, for example, digital palpating prior to refilling with a percutaneous needle by having a palpable ring structure surrounding the self-sealing diaphragm. In other embodiments the cover or diaphragm 112 may be designed such that a septum center may be located through one or more other methods. For example, the cover or diaphragm 112 may contain one or more radiopaque markers to allow for live x-rays to locate the fill port in real-time. The radiopaque markers may be, for example, tantalum, platinum, or gold, and may be embedded in a ring placed in the cover or diaphragm 112 in order to facilitate location of the center of the septum under X-ray. In some embodiments the separate material may be embedded using a unique pattern to further aid in visualization.

Furthermore, the cover or Bluetooth module/wireless RF charging platform 112 may protrude above the surrounding surface of housing 102, such that the diaphragm and “high-profile” design may be easily palpable under the skin to improve safety and efficacy of wireless connectivity. It should be appreciated that this is in contrast to a “low-profile” interskeletal design, where the functional component would have a smooth contour with normal bone and soft tissue all around, and would have a hearty muscle and fat soft tissue element covering it therefore presenting a soft tissue impediment to wireless charging and/or Bluetooth connectivity. The present functional component 100, however, may extend within the soft tissue implant with a high-profile design, and therefore allow, for example, more effective and safer wireless communication and/or charging. It may also be understood that the medical implant 100 may be constructed of materials that allow for ready visualization under ultrasound, for example the ring around or in the cover or diaphragm 112 may be constructed of a material having a different acoustic impedance from the rest of the body of the implant 100 and/or the septum center such that the difference in acoustic impedance between the materials allows for better visualization under ultrasound. As an example, in an embodiment the case of the implant 100 may be constructed of PEEK while the ring may be constructed of titanium or ceramics.

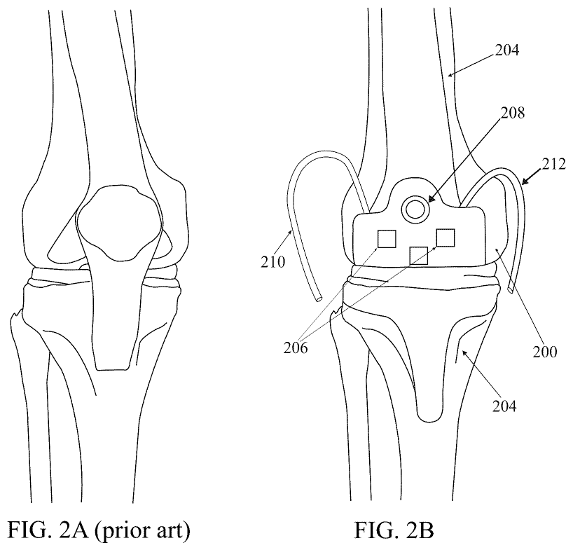

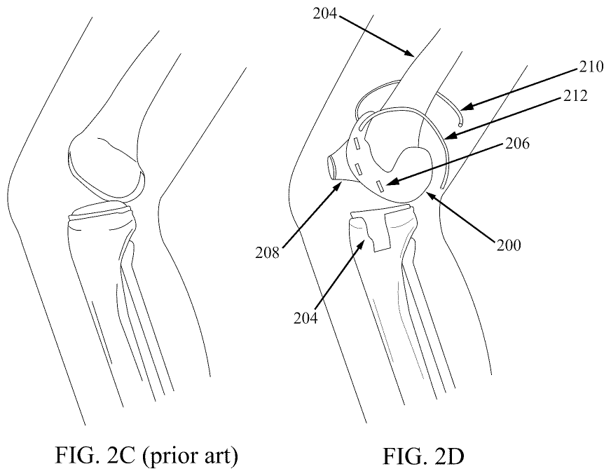

Referring to FIGS. 2A-5B generally, specific embodiments of an implantable pump and medical implants may now be described. It may be understood that these embodiments are exemplary and similar or same implants may be used in other embodiments. It may further be understood that any number of features described in one embodiment may be applicable in any other embodiment. It may be understood that the implantable pump may be a separate component from and/or may interface or replace any section of any of the implants as described.

Referring now to FIG. 2A-2D, FIG. 2A shows an anatomical knee and FIG. 2C shows a lateral view of an anatomical knee. FIG. 2B shows a knee replacement implant 200 according to an exemplary embodiment and FIG. 2D shows a lateral view of the knee replacement implant. It may be understood that in some embodiments the knee replacement 200 may replace hard and/or soft tissue of a patient. It may be understood that in instances of joint arthroplasty, bone and soft tissue have been resected to perform an index joint replacement. When the implants have been removed due to infection, the device may replace the resected bone and/or soft tissue. When the arthroplasty components remain in place, such as, for example, if an acute infection is being treated closer to the time of surgery, the device may replace the resected soft tissues (with or without a hard tissue component) and may have an extension design to allow standard fixation to nearby bone underneath. The exemplary knee replacement implant 200 may have functions such as those of the medical implant described in FIG. 1 above, may be attached to a patient, for example in a knee joint space 204 of a patient. In some embodiments the implant may be shaped so as to adequately replace bone and soft tissue in native and joint spaces. The design may further allow for necessary components to be appropriately housed and positioned, but still not occupy too much space within the joint. In some embodiments a high-profile, soft tissue reconstruction component may allow for a refillable diaphragm to be readily palpated and easily accessed with a simple, hypodermic needle. According to a specific embodiment, when a knee joint is replaced and the components then become infected, the resected bone and soft tissue from the initial procedure may be replaced, at least in part, but the described implant is used instead. In this embodiment, the replacement implant 200 may either be the permanent replacement to the current-day joint or spine implants, or may be a temporary replacement until a current disease or infection the patient is suffering from gets cured—thereby allowing revision surgery to then take place. It may be understood that in some embodiments the knee replacement implant 202 may be directly beneath the patient's skin 214 for easy needle access.

The medical implant may include one or more pump-assisted embedded components 206 and an injection port 208. The injection port 208 may allow for the filling or refilling of medication, cell therapies or other medicinal fluids to the medical implant 200 and may include soft tissue replacement. According to an embodiment the implant 200 may be positioned in the joint space between the two articulating bones using standard fixation techniques thereby minimizing micromotion in cases of being load-bearing. For example, In the case of the knee, the implant 200 may be located between the femur and tibia. By replacing the soft tissues between the bones, the implant may be readily palpable with so that it allows safe and easy medication refilling. In other embodiments, the implant may be secured not between the bones, but instead adjacent to one of the bones and the soft tissue by using a plate and screws, screws alone, staples, sutures and/or cement. In these embodiments soft tissue replacement may also allow the same access to refill medications. The implant 200 may be directly secured to bone, to soft tissue, or to both, using sutures, fixation plates, cement or tissue contours. The exact manner of device positioning and securing may be dependent on the particular nature of the boney and/or soft tissue loss in a particular case, as well as the anticipated activity level and weight-bearing status with the device in place.

The knee replacement implant 200 may further contain one or more catheters, for example a first catheter 210 and a second catheter 212, which may allow for medicine, cell therapies, or other medicinal fluids to be delivered to, for example, a joint space. In some embodiments the catheters may be directed to target the bones and/or soft tissues involved specifically in the pathologic process. For example, in an embodiment of treating a patient with an infected knee arthroplasty, one or more catheter(s) may be directed into the canal of the femur, one or more into the tibia and one or more into the soft tissue space of the knee joint. Medications to be delivered into these various spaces may include, for example, antimicrobials, antifungals, tumoricial medications, cell therapies and analgesics.

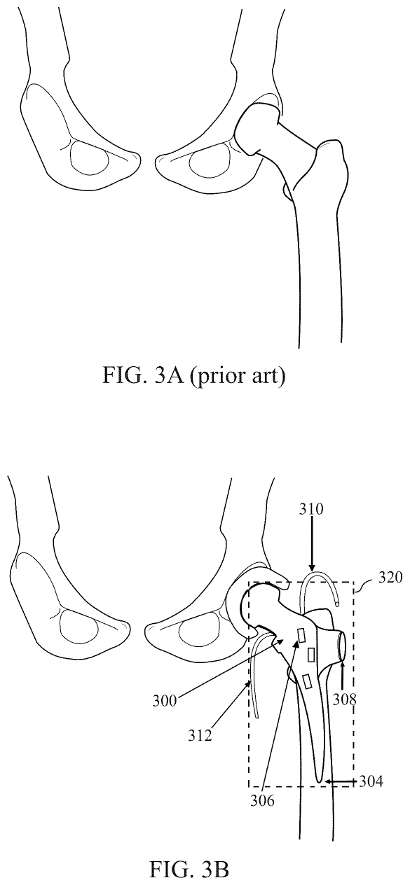

Referring now to FIGS. 3A-3B, FIG. 3A shows an anatomical hip and FIG. 3B shows a hip replacement implant 300. It may be understood that in some embodiments the hip replacement 300 may replace hard and/or soft tissue of a patient. The exemplary hip replacement implant 300 may have functions such as those of the medical implant described in FIG. 1 above and may be attached to a patient, for example in a hip space 304 of a patient. The hip implant 300 may have a functional design that allows this for combined hard (i.e. bone) and soft tissue (i.e. muscle/fat) implant to have embedded technology within (i.e. smart pump technology), and at the same time, also provide the patient standard mechanical support in line with current shapes and sizes on the market today in instances, for example, where an old implant has to be removed due to premature failure or infection. The medical implant may include one or more pump-assisted embedded components 306 and an injection port 308 which may extend to a level just under the skin. The injection port 308 may allow for the easy and quick filling or refilling of medication, cell therapies, and/or other medicinal fluids to the medical. The hip replacement implant 300 may further contain one or more catheters, for example a first catheter 310 and a second catheter 312, which may allow for medicine, cell therapies and/or other medicinal fluids to be delivered to, for example, a joint space. According to an embodiment, each catheter may include a catheter tip, and the catheter tips, at time of placement, may be strategically located and fixated to deliver therapeutic medicine to tissue-specific areas like bone, muscle and/or subcutaneous tissue areas - depending on the pathology being treated and/or source of infection. It may be understood that the pump and pump components may be fitted to the implant 300 according to the particular application, for example in area 320. The pump may be a variety of sizes and shapes, for example the size and shape as shown in the exemplary pump 100 above. It may be understood the size and shape of the implant 300 is for illustrative purposes only and is non-limiting.

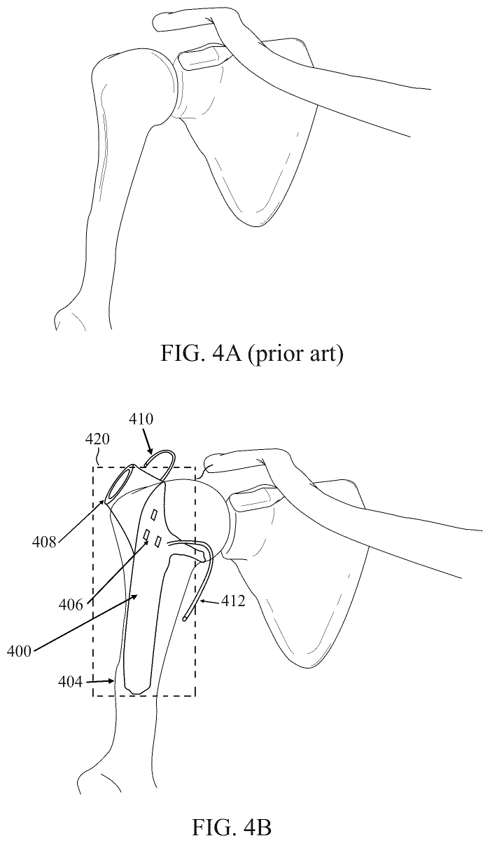

Referring now to FIGS. 4A-4B, FIG. 4A may show an anatomical shoulder and FIG. 4B may show a shoulder replacement implant 400. It may be understood that in some embodiments the shoulder replacement 400 may replace hard and/or soft tissue of a patient. The exemplary shoulder replacement implant 400 may have functions such as those of the medical implant described in FIG. 1 above and may be attached to a patient, for example in a shoulder space 404 of a patient. The shoulder replacement implant 400 may have a functional design that allows this for combined hard (i.e. bone) and soft tissue (i.e. muscle/fat) implant to have embedded technology within (i.e. smart pump technology), and at the same time, also provide the patient standard mechanical support in line with current shapes and sizes on the market today in instances, for example, where an old implant has to be removed due to premature failure or infection. The shoulder replacement implant 400 may include one or more “smart”, pump-assisted embedded components 406 and a high-profile injection port 408 that extends to a level just below the neighboring skin. The injection port 408 may allow for the easy and safe filling or refilling of medication, cell therapies or other medicinal fluids to the medical implant 400 and may include soft tissue replacement. The shoulder replacement implant 400 may further contain one or more catheters, for example a first catheter 410 and a second catheter 412, which may allow for medicine or other fluids to be delivered to, for example, a joint space. According to an embodiment, the catheter tips, at time of placement, may be strategically located and fixated to deliver therapeutic medicine to tissue-specific areas like bone, muscle and/or subcutaneous tissue areas - depending on the pathology being treated and/or source of infection. It may be understood that the pump and pump components may be fitted to the implant 400 according to the particular application, for example in area 420. The pump may be a variety of sizes and shapes, for example the size and shape as shown in the exemplary pump 100 above. It may be understood the size and shape of the implant 400 is for illustrative purposes only and is non-limiting.

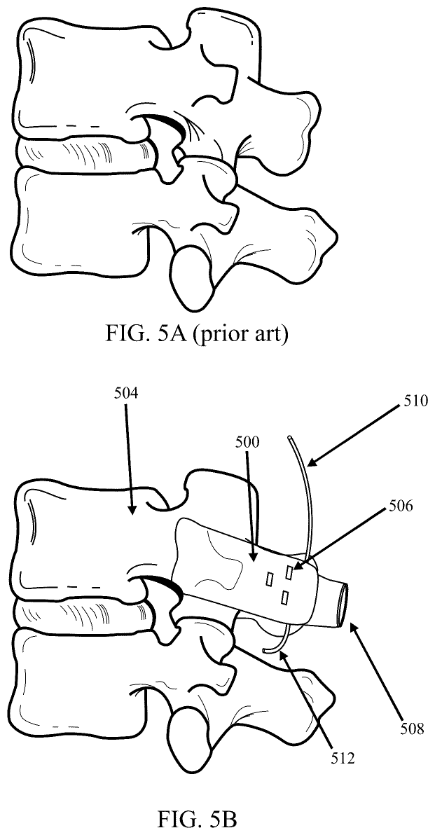

Referring now to FIGS. 5A-5B, FIG. 5A shows an anatomical spine and FIG. 5B shows a spine replacement implant 500. It may be understood that in some embodiments the spine replacement 500 may replace hard and/or soft tissue of a patient. The exemplary spine replacement implant 500 may have functions such as those of the medical implant described in FIG. 1 above and may be attached to a patient, for example in a spine space 504 of a patient. According to an embodiment the implant 500 design may allow for combined hard (i.e. bone) and soft tissue (i.e. muscle/fat) spine implant 500 to have embedded technology within (i.e. smart pump technology), and at the same time, also provide the patient standard mechanical support in line with current shapes and sizes on the market today in instances when an old implant has to be removed due to premature failure or infection. It may be understood that in the spine, the tissues replaced may be different from those in replaced joints depending on the exact level such as cervical, thoracic, lumbar, sacral or coccyx regions involved. In an embodiment the soft tissues replaced may include the vertebrae (bodies, laminae, spinous processes), the interspinous ligaments and/or the paraspinal musculature. The medical implant 500 may include one or more “smart” pump-assisted embedded components 506 and an injection port 508 that may extend to a level just below the skin. The injection port 508 may allow for the easy and safe filling or refilling of medication or other fluids to the medical implant 500 and may include soft tissue replacement. In an embodiment, the spine implant 500 may be placed via standard spine resection done in spinal column replacement therapy. In one or more embodiments the considerations for securing the device may be similar to those described for the knee and hip, with particular care being taken to not impinge upon neural elements of the dura and to minimize micromotion given the implants load-bearing status. The spine replacement implant 500 may further contain one or more catheters, for example a first catheter 510 and a second catheter 512, which may allow for medicine or other fluids to be delivered to, for example, a joint space. The catheter tips, at time of placement, may be strategically located and fixated to deliver therapeutic medicine to tissue-specific areas like bone, muscle and/or subcutaneous tissue areas—depending on the pathology being treated and/or source of infection. The catheters may also be directed to deliver medication into any part(s) or the vertebrae, the dural space, and involved soft tissues including paraspinal ligaments and overlying soft tissues.

As used in the paragraphs above smart pump technology may be understood to include technology such as, but not limited to, pressure sensors, temperature sensors, force sensors, and chemical sensing for conditions such as inflammation or infection.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A high-profile cranial medical implant comprising:
an injection port configured to be located under a patient's skin and configured to allow refilling of a fluid to the medical implant;
one or more pieces of smart, pump-assisted embedded technology;
one or more catheters configured to deliver the fluid from the medical implant to a soft tissue of the patient.

2. The medical implant of claim 1, wherein the one or more pieces of smart, pump assisted embedded technology are contained within an implant housing; and
the one or more pieces of smart, pump assisted embedded technology comprise:
a central processing unit; and
a rechargeable battery.

3. The medical implant of claim 2, wherein the one or more pieces of smart, pump assisted embedded technology further comprise:
a short range wireless module configured to allow wireless charging of the rechargeable battery; and
at least one propellant-driven or electro-osmotic pump.

4. The medical implant of claim 2, wherein the refillable reservoir is filled with one of medication, cell therapies or a medicinal fluid.

5. The medical implant of claim 1, wherein the replaced hard and/or soft tissue includes bone, muscle, and/or fat tissue.

6. The medical implant of claim 1, wherein the refillable reservoir is a high-profile soft tissue reconstruction reservoir and the injection port is configured to extend to just beneath a patient's skin.

7. The medical implant of claim 6, wherein the refillable reservoir is configured to be refillable via a hypodermic needle through the patient's skin.
